(19) United States
(12) Patent Application Publication
(10) Pub. No.: US 2005/0043513 A1
(43) Pub. Date: Feb. 24, 2005

(54) Mouse olfactory receptor gene superfamily

(76) Inventors: Stuart Firestein, New Milford, CT (US); Xinmin Zhang, Carlsbad, CA (US)

Correspondence Address: BAKER & BOTTS, 30 ROCKEFELLER PLAZA, NEW YORK, NY, 10112, US

(21) Appl. No.: 10/774,355

(22) Filed: Feb. 6, 2004

Related U.S. Application Data
(63) Continuation of application No. PCT/US02/25556, filed on Aug. 9, 2002.
(60) Provisional application No. 60/311,159, filed on Aug. 9, 2001.
(60) Provisional application No. 60/339,694, filed on Dec. 12, 2001.

(51) Int. Cl. C07K 14/705; C12Q 1/68; C07H 21/04

(52) U.S. Cl. 530/350; 435/6; 435/69.1; 435/320.1; 435/325; 536/23.5

(57) ABSTRACT

Olfactory receptors (ORs) encompass the largest gene superfamily in vertebrates. The present invention relates to data mining methods capable of identifying gene family members and particularly mouse OR (MOR) gene family members. The data mining method of the present invention has been used to identify 1296 mouse ORs from the nearly complete Celera mouse genome by a comprehensive data mining effort. MOR polypeptides, proteins, and nucleic acid molecules are disclosed. In addition to isolated, full-length MOR proteins, the invention further provides for MOR fusion proteins and anti-MOR antibodies. The invention also provides MOR nucleic acid molecules, recombinant expression vectors containing a nucleic acid molecule of the invention, host cells into which the expression vectors have been introduced and non-human transgenic animals in which a MOR gene has been introduced or disrupted. The MORs are useful for identifying compounds for treating anosmias, pest control and for developing better odorants, such as perfumes and food enhancers.

The present invention is a continuation of pending International Patent Application PCT/US02/25556 filed Aug. 9, 2002, published in English as International Publication No. WO03/094088 on Nov. 13, 2004, which claims priority to U.S. Provisional Patent Application Ser. Nos. 60/311,159, filed Aug. 9, 2001, and 60/339,694, filed Dec. 12, 2001, all of which are incorporated herein by reference in their entirety.

This invention was made with government support from US NIDCD and HFSP. Therefore, the government has certain rights in the invention.

### FIELD OF THE INVENTION

The present invention relates to the identification, isolation and characterization of mouse olfactory receptor (OR) polypeptides, the nucleic acids that encode them and methods of using the mouse ORs of the present invention.

### BACKGROUND OF THE INVENTION

The detection of environmental chemicals, commonly called odors, is mediated by peripheral olfactory organs of varied complexity in virtually all metazoans. Specialized sensory neurons initiate perception by detecting ambient molecules that interact with protein receptors in neuronal membranes. A pathway for olfactory signal transduction is initiated when the binding of odors to specific odor receptors activates specific G proteins. The G proteins stimulate a cascade of intracellular signaling events leading to the generation of an action potential which is propagated along the olfactory sensory axon to the brain. Odor receptors (ORs) have been identified as receptors that recognize odorant molecules. The ORs belong to the superfamily of seven transmembrane domain proteins, G-protein coupled receptors (GPCRs). A family of receptor guanylyl cyclases have been proposed as receptors for odors or pheromones. (Gibson, A. D. & Garbers, D. L., “Guanylyl cyclases as a family of putative odorant receptors,” Annu Rev Neurosci 23, pp.417-39 (2000)).

ORs were initially discovered in rat as a diverse multigene family characterized by seven transmembrane domain proteins. (Buck, L. & Axel, R. “A novel multigene family may encode odorant receptors: a molecular basis for odor recognition,” Cell 65, pp. 175-87. (1991)). They have since been identified in various species, including both invertebrates, e.g. nematode and fruit fly, and vertebrates, e.g. fish, amphibians, lizards, birds and mammals. (Mombaerts, P., “Seven-transmembrane proteins as odorant and chemosensory receptors,” Science 286, pp. 707-11 (1999)).

There are two classes of OR genes, class I (fish-like receptors) and class II (mammalian-like or tetrapod receptors). Structural analysis indicate that they differ in extracellular loop 3, which is likely to contribute to ligand specificity. (Freitag, J., Ludwig, G., Andreini, I., Rossler, P. & Breer, H. “Olfactory receptors in aquatic and terrestrial vertebrates,” J Comp Physiol [A] 183, pp. 635-50. (1998)). It has been hypothesized that class I receptors are activated by water-soluble odorants, whereas class II receptors are activated by volatile compounds. (Mezler, M., Fleischer, J. & Breer, H. Characteristic features and ligand specificity of the two olfactory receptor classes from Xenopus laevis. J Exp Biol 204, 2987-97. (2001)).

Class I receptors have been identified in fish and subsequently in the frog. (Ngai, J., Dowling, M. M., Buck, L., Axel, R. & Chess, A. “The family of genes encoding odorant receptors in the channel catfish,” Cell 72, pp.657-66. (1993); Freitag, J., Krieger, J., Strotmann, J. & Breer, H. “Two classes of olfactory receptors in Xenopus laevis,” Neuron 15, pp. 1383-92. (1995); Freitag, J., Ludwig, G., Andreini, I., Rossler, P. & Breer, H. “Olfactory receptors in aquatic and terrestrial vertebrates,” J Comp Physiol [A] 183, pp. 635-50. (1998)). Class I ORs had been previously thought to be evolutionary relics in mammals, however, a relatively large number of intact Class I ORs are found in the human genome. (Glusman, G., Yanai, I., Rubin, I. & Lancet, D. “The complete human olfactory subgenome,” Genome Res 11, pp. 685-702 (2001); Freitag, J., Ludwig, G., Andreini, I., Rossler, P. & Breer, H. “Olfactory receptors in aquatic and terrestrial vertebrates,” J Comp Physiol [A] 183, pp. 635-50. (1998)).

Class I ORs are appear to be prevalent in the mammalian genome, and may play important roles in mammalian olfaction. Class I ORs are expressed in the most dorsal zone of the olfactory epithelium. (Bulger, M. et al. “Conservation of sequence and structure flanking the mouse and human beta-globin loci: the beta-globin genes are embedded within an array of odorant receptor genes.,” Proc Natl Acad Sci USA 96, pp. 5129-34. (1999); Conzelmann, S. et al. “A novel brain receptor is expressed in a distinct population of olfactory sensory neurons,” Eur J Neurosci 12, pp. 3926-34. (2000); Malnic, B., Hirono, J., Sato, T. & Buck, L. B. “Combinatorial receptor codes for odors,” Cell 96, pp. 713-23. (1999); Raming, K., Konzelmann, S. & Breer, H. “Identification of a novel G-protein coupled receptor expressed in distinct brain regions and a defined olfactory zone,” Receptors Channels 6, pp.141-51(1998)).

Class II receptors are primarily found in tetrapod species. These receptors have been found in all four zones of the olfactory epithelium. In mammals, the olfactory epithelium appears to be organized into distinct topographic regions or zones in which expression of a particular receptor gene appears to be restricted to one of the four zones in the epithelium. (Ressler et al. “A zonal organization of odorant receptor gene expression in the olfactory epithelium,” Cell 73, pp.597-609 (1993); Vasser et al. “Topographic organization of sensory projections to the olfactory bulb,” Cell 79, pp.981-991 (1994)). In addition, it each neuron expresses only one or a few odorant receptors.

In mammals, ORs constitute the largest gene superfamily in the genome. It has been estimated that there are approximately 1000 ORs in the mouse and rat, approximately 500-750 ORs in human, and approximately 100 ORs in fish. (Buck, L. B. “Information coding in the vertebrate olfactory system,” Annu Rev Neurosci 19, pp.517-44 (1996) and Mombaerts, P. “Molecular biology of odorant receptors in vertebrates,” Annu Rev Neurosc 22, pp. 487-509 (1999)).

Sequence information relating to OR genes has been obtained through a variety of strategies, including the use of degenerate PCR primers on cDNA or genomic DNA templates. These efforts have resulted in gene fragments with a small number of full-length genes recovered. Both primer bias and possible recombination among highly related sequences lead to the failure to detect many genes. (Glusman, G. et al. “Sequence, structure, and evolution of a complete human olfactory receptor gene cluster,” Genomics 63, pp. 227-45. (2000); Glusman, G., Clifton, S., Roe, B. & Lancet, D. “Sequence analysis in the olfactory receptor gene cluster on human chromosome 17: recombinatorial events affecting receptor diversity,” Genomics. 37(2), pp.147-60 (1996)). As a result, relatively few OR gene sequences have been identified.

The availability of genome sequences has lead to the developmental of various analytical approaches for identifying members of gene families. Public databases containing nearly complete human, mouse, rat, Drosophila, and yeast genomes are available for data mining. Keyword and homology based searches applying various algorithms have been used to identify gene and protein families. As an example, U.S. Pat. No. 6,395,889 discloses a method of identifying protease family members from various protease families using known consensus motifs in TBLASTN searches or Hidden Markov Model (HMM) motifs or both. This patent discloses the application of HMM to generate consensus sequences specific for protease family members. The consensus sequences are used as queries in TBLASTN searches.

Following the release of the human genome, the human OR repertoire was explored at the whole genome level. (Glusman, G., Yanai, I., Rubin, I. & Lancet, D. “The complete human olfactory subgenome,” Genome Res 11, pp. 685-702 (2001); Zozulya, S., Echeverri, F. & Nguyen, T. “The human olfactory receptor repertoire,” Genome Biol 2, pp.1-12 (2001)). Approximately 900 ORs, distributed within 24 clusters throughout the genome (except chromosome 20 and Y), were discovered in human, with a 60% of these apparently being pseudogenes. It is hypothesized that the degeneration of ORs in humans is due to the smaller role of olfaction in comparison to vision and hearing.

In contrast to the fewer than 350 intact OR genes found in human, mice are thought to have a much larger repertoire of ORs. Since mice have become the experimental animal of choice in olfactory studies, due largely to the success of gene targeting in mice, the complete mouse OR repertoire would therefore be desirable. Until recently only ˜100 full-length mouse OR sequences were available in the public database, with more than half of these obtained by sequencing genomic regions of known OR gene clusters. (Xie, S. Y., Feinstein, P. & Mombaerts, P. “Characterization of a cluster comprising approximately 100 odorant receptor genes in mouse,” Mamm Genome 11, pp. 1070-8 (2000); Hoppe, R., Weimer, M., Beck, A., Breer, H. & Strotmann, J. “Sequence analyses of the olfactory receptor gene cluster mOR37 on mouse chromosome 4,” Genomics 66, pp. 284-95 (2000); Lane, R. P. et al. “Genomic analysis of orthologous mouse and human olfactory receptor loci,” Proc Natl Acad Sci USA 98, pp. 7390-5. (2001)). Thus, a whole genomic approach is likely the most efficient and thorough means to retrieve the complete OR repertoire.

In May 2001, the Celera mouse genome was released (www.celera.com). The present invention addresses the need for identifying the complete mouse OR repertoire by providing novel data mining methods. This method was used with the nearly complete Celera mouse genome to identify the mouse OR repertoire which comprises of approximately 1300 ORs.

### SUMMARY OF THE INVENTION

The present invention relates to a data mining method of identifying candidate gene sequences. The data mining method of the present invention uses the novel combination of a low stringency TBLASTN search and Hidden Markov Model (HMM) profiles designed from known OR sequences to identify putative OR candidates for further analysis. The low stringency TBLASTN search allows for the identification of a greater pool of OR candidates.

In another aspect, the present invention consists of MOR (mouse olfactory receptor) nucleic acid sequences identified using the method of the present invention (DNA and RNA), MOR proteins and peptides, and antibodies raised against the MOR proteins. The MOR nucleic acid sequences, proteins and peptides of the present invention are useful for identifying compounds which can be used to treat anosmias, for pest control and for the development of deodorants.

In still another aspect, the present invention consists of host cells and animals genetically engineered to express MOR proteins and peptides, DNA vectors that contain any of the foregoing MOR nucleic acid sequences and/or their complements (i.e., antisense), and DNA expression vectors that contain any of the foregoing MOR nucleic acid sequences operatively linked to a regulatory element that directs the expression of the MOR nucleic acid sequences.

### BRIEF DESCRIPTION OF THE DRAWINGS

The present invention may be better understood from the following detailed description of exemplary embodiments with reference to the attached drawings in which:

FIG. 1 shows a phylogenetic tree of Mouse Olfactory Receptor families, where FIG. 1 shows a phylogenetic tree of the consensus sequences of mouse OR families in which bootstrap values are shown at nodes with >50% support and the tree was rooted using human and mouse melanocyte stimulating hormone receptors (MSH-R); and FIGS. 1a-c shows a list of all the families in the same order as in a) (1-41 are Class I families, 101-286 are Class II families).

FIGS. 2a and 2b show the chromosomal distribution of Mouse OR Genes, where FIG. 2a depicts mouse chromosomes, drawn according to the Celera scaffold assembly, showing the number of ORs at various positions along the chromosomes; and FIG. 2b is a graph showing the number of intact genes (dark) and pseudogenes (shaded) on each chromosome.

FIG. 3 is a schematic diagram demonstrating the distribution of OR Clusters.

FIG. 4 is a schematic diagram showing the sequence logos for the Open Reading Frames of Class I and Class II ORs.

FIG. 5 shows the unrooted phylogenetic tree of human and mouse ORs, where FIG. 5a shows an unrooted phylogenetic tree of the consensus sequences of mouse and human OR families; and FIG. 5b shows an unrooted phylogenetic tree of the intact full length ORs belonging to families in the group shaded in 5a; and FIG. 5c shows an unrooted phylogenetic tree of the intact full length ORs of the Iva1 ORs (Mouse OR families 258 and 259) and families close to them.

FIG. 6 is a flow diagram of the data mining method of the present invention.

FIGS. 7a and 7b are flow diagrams of an exemplary embodiment of the data mining method of the present invention, where FIG. 7a is a flow diagram of a profile HMM OR classifier; and FIG. 7b is a flow diagram of mouse OR data mining.

### DETAILED DESCRIPTION OF THE INVENTION

In accordance with the present invention, a novel data mining method for identifying candidate genes in a gene family is provided. In a preferred embodiment of the invention, candidate MOR genes are identified using the data mining method of the invention from mouse genomic databases.

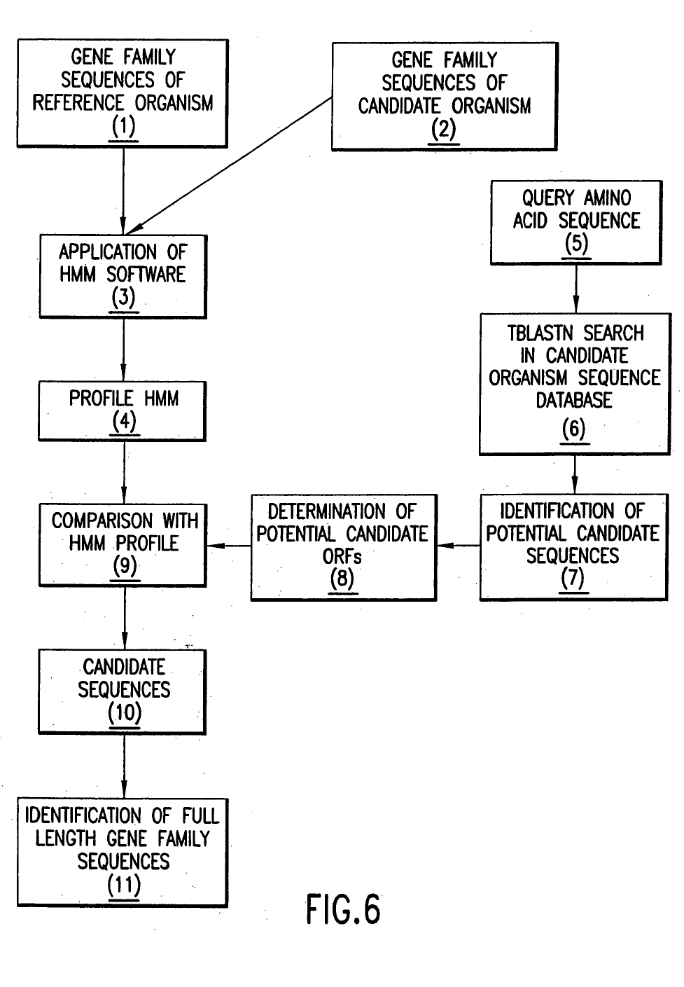

Referring to FIG. 6, known sequences from a gene family 1 that has been well characterized in a reference organism may be used together with a limited number of known sequences of the same gene family from a candidate organism 2. To obtain gene sequences of the reference and candidate organism, homology-based searches are conducted. A variety of known algorithms are disclosed publicly and a variety of publicly and commercially available software can be used. Examples include, but are not limited to, MacPattern (EMBL), BLASTN(NCBI), BLASTX(NCBI) and FASTA (University of Virginia). Sequences can be extracted from public databases, such as the National Center for Biotechnology (NCBI), Human Olfactory Receptor Data Exploratorium (HORDE), the Olfactory Receptor Database (ORDB), and mouse EST database. In a preferred embodiment of the invention, full length MAMMALIAN OR database, a collection of all the known full length human and mouse ORs from HORDE, ORDB and Genbank, and non-OR GPCR database, a collection of GPCRs excluding ORs from SWISS_PROT Annotated Protein Sequence Database, are used. According to the invention, any DNA sequence database may be used in the data mining method of the present invention.

After having obtained the reference and candidate organism gene family sequences, a full characterization of the gene family is conducted. This may be accomplished by applying a Hidden Markov Model (HMM) to the reference organism and candidate organism gene family sequences 3 to build a HMM profile 4. HMM 3 is a probabilistic approach which analyzes consensus primary structures of gene families. (See Eddy, S. R. Curr Opin Str Bio 6:361-365 (1996); Durbin, R., S. R. Eddy, et al. (1998). “Biological sequence analysis: probalistic models of proteins and nucleic acids”. Cambridge, UK, Cambridge University Press; Eddy, S. R. (1998). “Profile hidden Markov models.” Bioinformatics 14(9): 755-63.)

To facilitate the building of HMM profiles, an alignment software program, such as ClustalW or Clustal X, may be used to align the gene family sequences from the reference organism. Alternatives to ClustalW include, but are not limited to, MultAlin, MSA (Multiple Sequence Alignment), DIALIGN, and MACAW (Multiple Alignment Construction & Analysis Workbench). (Corpet, F. “Multiple sequence alignment with hierarchical clustering” 1988, Nucl. Acids Res., 16 (22), 10881-10890; Lipman, D J, Altschul S F, Kececioglu J D: “A Tool for Multiple Sequence Alignment.” Proc. Natl. Acad. Sci. USA 86:4412-4415, 1989; B. Morgenstern, K. Frech, A. Dress, T. Werner: DIALIGN: Finding local similarities by multiple sequence alignment. Bioinformatics, 14, 1998, 290-294). The alignment produced may then be used to build the profile HMMs. More than one profile HMM may be built if the gene family contains subfamilies. A hmmsearch program found in the HMMER software package can be used to generate the profile HMM. Other non-limiting examples of alignment software include Sequence Alignment and Modeling System (SAM) (UC Santa Cruz) and HMMpro (NetID, Inc.).

Again referring to FIG. 6, query sequences 5 (which may be amino acid sequences identified as gene family members of the reference organism or the candidate organism, or may be any amino acid sequence of the gene family from any organism identified by any means known in the art) are subjected to a TBLASTN search 6 (http://www.ncbi.nlm.nih.gov/BLAST/blast_overview.html) against a candidate organism sequence database (which preferably comprises a nearly complete set of sequence information from the genome of the candidate organism, but can be incomplete). In one embodiment, candidate nucleic acid sequences are obtained using known reference organism sequences as query sequences and applying a TBLASTN search to mine a nucleic acid database of the candidate organism. A TBLASTN search 6 allows for the identification of candidate nucleic acid sequences 7 by comparing a query amino acid sequence 5 to nucleic acid sequence databases that are dynamically translated into amino acid sequences. The TBLASTN search is conducted using low threshold or low stringency values to obtain a large pool of potential candidate sequences. In a preferred embodiment of the invention, an E-value of <10 is used in the TBLASTN search and all hits with an E-value <10 are collected. An E-value of <5 may also be used. Potential candidate open reading frames (ORFs) are then determined 8. Any program known in the art capable of translating DNA into protein can be used to determine potential candidate ORFs. As a non-limiting example, programs such as ORF finder (http://www.ncbi.nlm.nih.gov/gorf/gorf.html) maybe used.

The potential candidate ORFs obtained are compared with the profile HMMs 9 to obtain candidate sequences 10. In addition, the potential candidate sequences may be classified into subgroups corresponding to subfamilies during the comparison step.

The method may optionally comprise determining putative secondary structures of the potential candidate ORFs as described below in Example 1. These structures may be determined using the PredictProtein Server, (http;//cubic.bioc.columbia.edu/predictionprotein/) or the TMHMM server (http://www.cbs.dtu.dk/services/TMHMM/).

If candidate sequences are identified as putative pseudogenes (i.e. fragments, disruptions, etc.) they may optionally be subjected to conceptual translation using FASTY3 (http://http.virginia.edu/pub/fasta/). This algorithm aligns query DNA sequences with protein sequences and takes into consideration the possibility of frame shifts and premature stop codons to identify the putative undisrupted sequence of the gene. Candidates identified can also be subjected to BLAST searches through databases comprising only known genes from that gene family should the database exists.

The various identified candidate sequences may then be subjected to further analysis incorporating ORF discovery, profile HMM searches and BLASTP searches in an iterative process to determine full length sequences 11. Exhaustive iterative TBLASTN searching is preferably continued until no new candidate sequences are found.

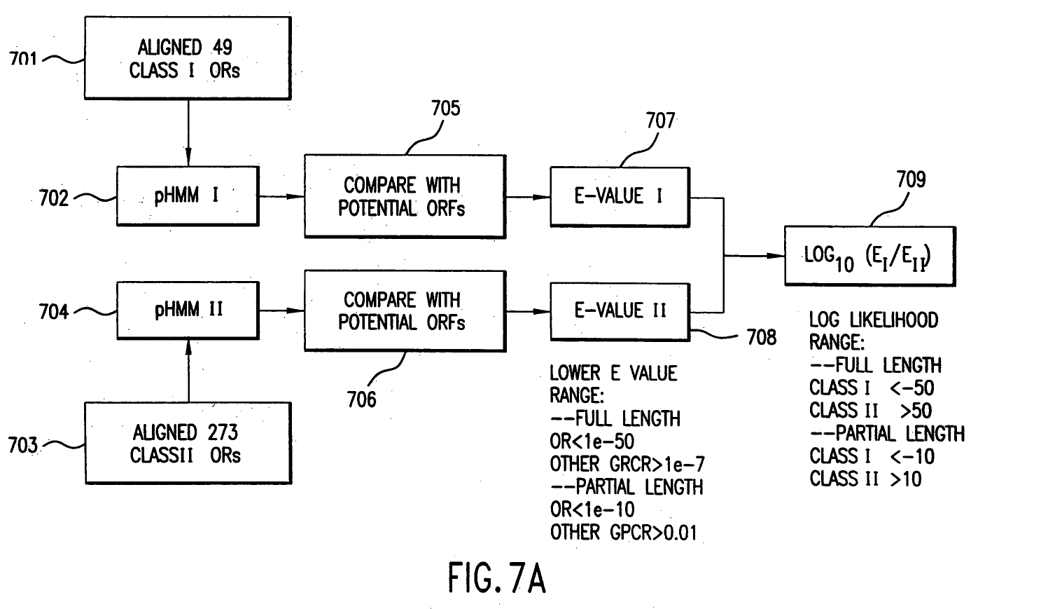

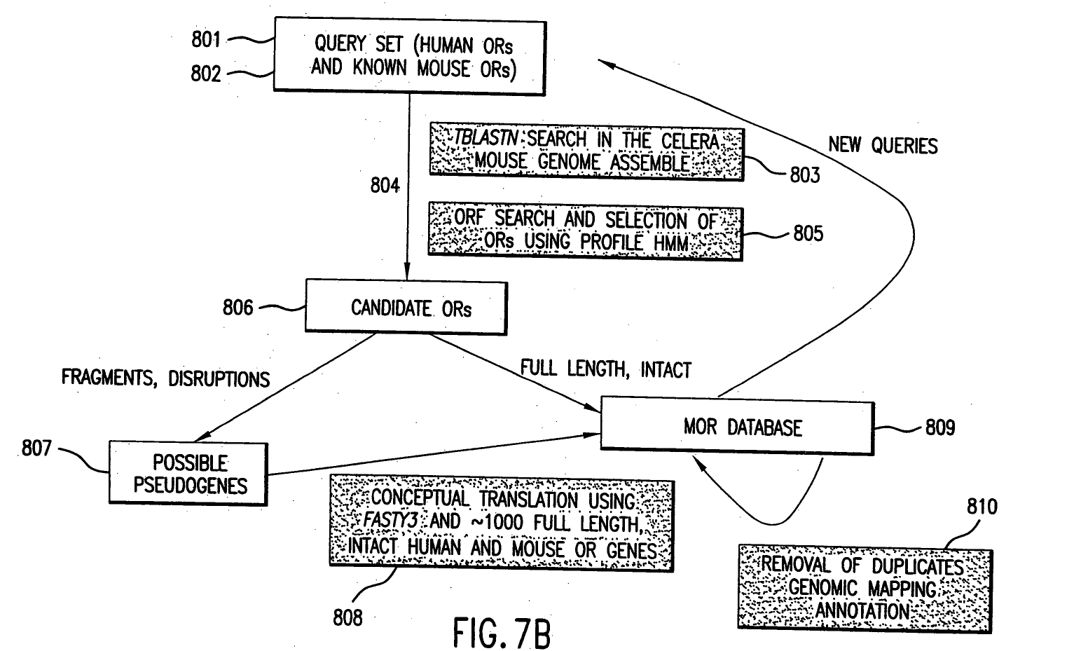

FIGS. 7A and 7B are flow diagrams showing a preferred embodiment of the data mining method of the invention. Referring to FIG. 7B and described in Example 1, a TBLASTN search 803 for candidate OR sequences was applied to known reference organism (human) ORs 801 as query sequences. Alternatively, mouse sequences 802 may be used as the query sequences. The TBLASTN search 803 was conducted using low threshold or low stringency values (E-value <10) to obtain a large pool of potential candidate sequences by collecting all hits with an E-value <10. Potential ORFs were then determined 804. Any program known in the art capable of translating DNA into protein can be used to determine potential candidate ORFS. As a non-limiting example, programs such as ORF finder (http://www.ncbi.nlm.nih.gov/gorf/gorf.html) may be used.

Referring to FIG. 7A, known human and mouse OR sequences were subject to alignment using Clustal W and divided into class I ORs 701 and class II ORs 703. The class I ORs 701 and class 2 ORs 703 were used to build two profile HMMs, pHMMI 702 and pHMMII 704. This is further descried in detail in Example 1 below. The profile HMMs were built using the hmmsearch program found in the HMMER software package. These HMM profiles were compared with potential candidate ORs 705 and 706. E-values were determined for both comparisons 707 and 708, respectively. Log likelihood ratio (Log₁₀EI/EII) were then calculated 709. OR/non-OR was determined by the smaller E-value and ClassI/ClassII was determined by the log likelihood ratio as further discussed below in Example 1.

Again referring to FIG. 7B, potential candidate ORs 806 were obtained by comparison with the HMM profiles 805 obtained as described above and shown in FIG. 7A.

The method may optionally comprise determining putative secondary structures of the potential candidate OR ORFs as described below in Example 1. These structures may be determined using the PredictProtein Server, (http;//cubic.bioc.columbia.edu/predictionprotein/) or the TMHMM server (http://www.cbs.dtu.dk/services/TMHMM/) to obtain mouse OR sequences. The putative secondary structures of the potential candidate OR ORFs are then evaluated for structural characteristics of OR proteins, i.e. seven transmembrane domains, N terminal domain length of 10-40 amino acids, and C terminal domain length of 7-30 amino acids.

Candidate ORs were evaluated to determine whether they represented putative pseudogenes (i.e. fragments and/or disruptions) or full length sequences. Full length sequences were directly classified as sequences of the MOR database 809 and putative pseudogenes were subjected to conceptual translation using FASTY3 (http://http.virginia.edu/pub/fasta/) 808 to identify whether they represented full length sequences. The FASTY3 algorithm aligns query DNA sequences with protein sequences and takes into consideration the possibility of frame shifts and premature stop codons to identify the putative undisrupted sequence of the gene. Alternatively, putative undisrupted sequence can be obtained using FASTX3. Putative pseudogenes identified can also be subjected to BLAST searches through databases comprising only known genes from that gene family should the database exists. Any full length sequences identified from possible pseudogenes were also classified as sequences of the MOR database. The MOR database can be refined by the removal of duplicate sequences 810.

The various identified candidate sequences which were classified as members of the MOR database 809 were then be subjected to further analysis through iteration to further refine and develop the MOR database. Exhaustive iterative TBLASTN searching is preferably continued until no new candidate sequences are found.

The genomic organization of identified OR sequences were mapped and genomic or chromosomal localization of mapped OR sequences were evaluated for phylogenetic similarity as discussed in Example 5.

The invention further contemplates identifying any remaining MOR sequences as the mouse genomic databases become more complete.

Using the data mining method of the present invention, a nearly complete mouse olfactory receptor repertoire consisting of 1296 MOR genes (SEQ ID NOS: 1-1296) and 1296 MOR proteins (SEQ ID NOS: 1297-2592) was identified and characterized as discussed in Example 4.

Accordingly, another aspect of the present invention is directed to mouse olfactory receptor (MOR) nucleic acid sequences that encode MOR proteins, mutant MOR proteins, peptide fragments of MOR proteins, truncated MOR, and MOR fusion proteins. The sequences are set forth as SEQ ID NOS: 1-1296 in the attached sequence listing. These include, but are not limited to, nucleotide sequences encoding polypeptides or peptides corresponding to the transmembrane and/or cytoplasmic domains of MOR or portions of these domains. Of these sequences, SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296 have not been previously identified.

Alternative to the above-described method, MOR nucleotide sequences may be identified using a variety of different methods known to those skilled in the art. For example, a cDNA library constructed using RNA from a tissue known to express MOR can be screened using a labeled MOR probe. A genomic library may be screened to identify nucleic acid molecules encoding a MOR protein. Further, MOR nucleic acid sequences may be derived using PCR with two oligonucleotide primers designed on the basis of the MOR nucleotide sequences disclosed herein.

The present invention also contemplates DNA vectors that contain any of the foregoing MOR nucleic acid sequences and/or their complements, i.e., antisense. The DNA expression vectors may contain any of the foregoing MOR nucleic acid sequences operatively linked to a regulatory element that directs the expression of the MOR coding sequences. As used herein, regulatory elements include but are not limited to inducible and non-inducible promoters, enhancers, operators and other elements known to those skilled in the art that drive and regulate expression, as well as The present invention also provides for viral vectors comprising any of the foregoing MOR sequences and/or their complements. The present invention provides for proteins and peptides encoded by MOR nucleic acid sequences as set forth in SEQ ID NOS: 1297-2592. Of these sequences, SEQ ID NOS: 1300-1302, 1302, 1308-1310, 1312-1323, 1326-1346, 1348, 1349, 1351-1365, 1368-1382, 1384-1388, 1393-1396, 1398-1417, 1419, 1421, 1423-1440, 1442-1448, 1450-1460, 1463, 1464, 1467-1469, 1471-1478, 1480-1487, 1489, 1490, 1492-1501, 1503, 1505-1510, 1512-1515, 1517-1521, 1523-1541, 1546-1548, 1550, 1552-1556, 1560-1566, 1569-1577, 1581-1588, 1590, 1592-1596, 1599, 1600, 1602-1609, 1611-1614, 1616, 1618-1625, 1627-1631, 1635-1648, 1650, 1651, 1653-1684, 1687, 1689-1722, 1724, 1725, 1728-1764, 1766-1782, 1784-1791, 1793-1803, 1805-1809, 1811-1821, 1823, 1824, 1828, 1829, 1831, 1834, 1836-1838, 1841-1852, 1854-1859, 1861-1872, 1874-1893, 1895-1914, 1916, 1920-1924, 1926, 1927, 1933-1952, 1955-1958, 1961, 1963-1971, 1973-1979, 1981-1993, 1997, 2000-2012, 2015-2034, 2036-2053, 2055-2062, 2064-2080, 2082, 2085-2092, 2096, 2097, 2099-2108, 2110-2130, 2132-2161, 2163-2222, 2224-2356, 2358-2390, 2392-2411, 2413-2422, 2425-2431, 2434-2440, 2442-2452, 2455-2461, 2464-2467, 2469-2471, 2473-2488, 2490-2492, 2494-2497, 2499, 2501-2523, 2525, 2528-2546, 2548, 2549, 2551-2554, 2556-2564, 2568-2570, 2572-2589, 2591 and 2592 have not been previously described. In addition, the present invention also contemplates mutant MOR proteins, peptide fragments of MOR proteins, truncated MOR, MOR fusion proteins, and obvious variants of these peptides that are within the art to make and use. Fusion proteins may include but are not limited to full length MOR, truncated MOR or peptide fragments of MOR fused to another protein or peptide such as a marker protein. MOR peptides and proteins may be synthesized or produced using recombinant DNA technology well known in the art.

Another aspect of the present invention relates to antibodies. The polypeptides of the invention or their fragments, or cells expressing them, can be used as immunogens to produce monoclonal or polyclonal antibodies that are specific for polypeptides of the present invention. Antibodies generated against polypeptides of the present invention may be obtained by administering the polypeptides or fragments, or cells expressing them to an animal, preferably a non-human animal, using protocols familiar to one skilled in the art. For preparation of monoclonal antibodies, any technique known to one skilled in the art which provides antibodies produced by continuous cell line cultures can be used.

The present invention provides for methods for detecting the presence of a MOR nucleic acid molecule, protein or polypeptide in a sample by contacting the sample with an agent capable of detecting a MOR nucleic acid molecule, protein or polypeptide such that the presence of a MOR nucleic acid molecule, protein or polypeptide is detected in the biological sample. The agent may include, but is not limited to, a nucleic acid probe comprising sequences from MOR genes, an antisense molecule, and an antibody.

The present invention also provides for genetically engineered host cells that contain any of the foregoing MOR sequences operatively linked to a regulatory element that directs the expression of the MOR coding sequences in the host cell.

The invention also provides a method for producing a MOR protein by culturing in a suitable medium, a host cell of the invention, such that the MOR protein is produced.

The present invention further provides for transgenic mice or other non-human organism that contain any of the foregoing MOR sequences.

In a further aspect of the present invention, there is provided a method of screening compounds to identify those that stimulate or inhibit the biological function of the polypeptides of the present invention. In addition, the present invention provides for a method of screening compounds to identify those that regulate the expression level of the polypeptide. Such methods identify agonists or antagonists that may be employed for therapeutic, cosmetic or other purposes.

The MOR proteins of the present invention can be used to screen a compound for the ability to stimulate or inhibit interaction between the MOR receptors and a target protein that normally interacts with the MOR receptor. The target can be any protein with which the receptor normally interacts, including, but not limited to, extracellular ligands and intracellular signaling proteins. The assay includes the steps of combining the MOR receptor with a candidate compound under conditions that allow the MOR receptor or a fragment of the MOR receptor to interact with the target protein, and to detect the formation of a complex between the MOR receptor and the target or to detect the biochemical consequence of the interaction with the MOR receptor and the target, such as detecting the induction of a reporter gene (comprising a target-responsive regulatory element operatively linked to a nucleic acid encoding a detectable marker, e.g., luciferase or GFP), detecting a cellular response, e.g., development, differentiation or rate of proliferation, and detection of the activation of a substrate, or change in substrate levels.

For example, agonists or antagonists of the MOR polypeptides may be designed for treatment of mammalian anosmias, i.e., to treat a subject that has lost the ability to smell. The present invention provides for the identification of genes relating to mammalian anosmias and the development of treatments for anosmias. In addition, more effective perfumes may be designed for cosmetic purposes. Agents may be identified from a variety of sources, for example, cells, cell-free preparations, and natural product mixtures derived from microorganisms, plants or animals. The agents may be naturally occurring or synthetic, and may be a single substance or a mixture. Screening may be performed in high throughput format using chemical libraries, combinatorial libraries, expression libraries and the like.

Compounds useful of the treating anosmias may be identified by using behavioral assays in an anosmic mouse and determining whether the anosmic mouse can detect isovaleric acid at levels below 10⁻⁵M and preferably below 10⁻⁶M. Osmic mice can detect isovaleric acid at 10⁻⁷M. A conditioned avoidance assay can be used, where mice that can smell isovaleric acid are conditioned to associate the smell with an aversive stimulus. (Griff and Reed. “The Genetic Basis for Specific Anosmia to Isovaleric Acid in the Mouse.” Cell 83:407-414 (1995)).

The present invention provides for methods of screening for ligands of the MOR receptors. In one embodiment of the present invention, identified ligands for MOR receptors can be used to design more efficient compounds for stimulating or inhibiting MOR receptor function.

Agents which stimulate olfactory receptors can cause an animal to become attracted to a particular substance. Identification of stimulatory agents may allow one to design more efficient compounds for pest control, i.e., rodent attraction to a poisonous substance. This may be selective to pests, i.e. compounds may be chosen that stimulate ORs not found in humans. Furthermore, one may be able to design more appealing fragrances for human use in cosmetics, food, and household products by the identification of compounds that stimulate MORs that have human homologues.

Agents which stimulate olfactory receptors can also cause an animal to become repelled by a particular substance. These compounds may be used in pesticides or extermination. For example, these agents may be useful to develop “animal friendly” exterminants which simply deter the animal from entering a home or a yard. They may also be used to deter a pet from certain activities. For example, an agent whose odor can be detected by a pet, but not its owner, may be useful to deter the pet from sitting on furniture or scratching furniture without being offensive to the owner. Such agents may also be used in place of a fence to keep a pet in the yard or to keep unwanted pets or pests out of the yard.

The invention further provides for a method of identifying compounds for treating anosmia comprising introducing a candidate compound to an anosmic animal that is only able to detect isovaleric acid at concentrations higher than 10-5 M, observing the animal for an indication that it detected isovaleric acid through a behavioral assay.

### EXAMPLES

The invention is further described by the Examples below. These examples describe and demonstrate embodiments within the scope of the present invention. The examples are given solely for the purpose of illustration and are not construed as a limitation of the present invention, as many variations thereof are possible without departing from the spirit and scope of the invention.

### Example 1

### Data Mining

An exhaustive TBLASTN search incorporating profile HMM (Hidden Markov models) search was used to obtain all the possible OR sequences from the Celera mouse genome using, for example, the data mining method represented by the flow diagram of FIG. 6. Human intact ORs were aligned to build profile HMMs and these models were utilized to search for mouse candidate OR genes. Conceptual translation was used to recover the original ORFs for possible pseudogenes. Duplicates were removed and resulting OR genes were mapped to genomic locations according to the mapping data of the scaffolds by Celera.

Methods

Profile HMM Classifier

Referring to FIG. 7a, all intact human OR protein sequences from the HORDE database (http://bioinformatics.weizmann.ac.il/HORDE) were aligned using ClustalW, and the alignments were used to build two profile HMMs (one for 49 Class I ORs 4c, the other for 273 Class II ORs 4c′) using the HMMER software package (http://hmmer.wustl.edu/). New query sequences were compared with both models. As shown in FIG. 7a, the output E-values and the log likelihood ratio (logloEr/Erv) were used to classify the query sequences as OR/non-OR (one of the E-values must be less than 1-1°), and to further classify into classes if they are ORs (log likelihood ratio>10, Class II; <10, Class I; otherwise uncertain). Cross-validation experiments showed that models based on a 40-sequence training set could predict ORs and OR classes with high specificity and sensitivity for both full-length and fragmental ORs. Using a HMM classifier allows for the coupling of a low-stringency TBLASTN search to get a large number of hits, then choose the candidate ORs by comparing them to the profile HMMs. This is more powerful than previously known methods because a greater number of candidate sequences is created, which improves the chances of finding more genes within the family.

Data Mining in the Mouse Genome

Referring to FIG. 7b, representative human OR sequences 1a (which generally have <40% pairwise protein identity) and mouse OR sequences 2a were used as query sequences for a TBLASTN search 6a in the Celera mouse genome. All hits with an E-value <10 were collected. For each hit, a 4-Kb genomic region covering the hit position was used to search for the longest ORF 8a in that region. The translated protein sequences from these ORFs were then sent to the profile HMM classifier 9a to obtain the E-values and log likelihood ratio. These sequences were also subjected to a BLASTP search against two databases: the mammalian OR database (including all the known human and rodent ORs) and a non-OR GPCR (−400 genes) database. The results from both the profile HMM classifier and the BLASTP search were used to classify a query as OR or non-OR. In total, 1,405 potential OR sequences were found. All steps, except the TBLASTN search, were automated by scripts written in MATLAB (the MathWorks, Inc., Natick, Mass.).

Selection of Full-Length ORs.

The secondary structures of all potential ORs longer than 275 amino acids were obtained (from the TMHMM server http://www.cbs.dtu.dk/services/TMHMM-2.0/ or the PredictProtein server http://cubic.bioc.columbia.edu/predictprotein/, or by alignment to other ORs with secondary structure already predicted). If a potential OR had 7 transmembrane domains and the N and C terminals had the appropriate length (N 10-40 aa, C 7-30 aa), it was treated as a full-length mouse OR and was added into the mouse OR database directly. By these criteria, 759 full-length mouse ORs were discovered out of the 1,405 sequences (4 were removed later as duplicates).

Conceptual Translation.

All other sequences (including 199 with terminals that were too long or short, and 447 consisting of only a short fragment) were subjected to conceptual translation using FASTY (http://http.virginia.edu/pub/fasta/). FASTY aligns the query DNA sequence with a specified protein database considering possible frame shifts and premature stop codons. The full-length OR protein database used by FASTY includes 347 intact human ORs and the 755 full-length mouse ORs previously identified. The resulting conceptual translation was based on the best hit, but extended at both ends according to the top 20 FASTY hits. This method also ensures that the best translation starting points were chosen for ORs with multiple methionines in the N terminus. From conceptual translation, an additional 129 full-length intact ORs were discovered from the 199 ORs that previously had terminals considered either too long or too short. For these ORs, either the best translation starting points were determined by conceptual translation or the conceptual translation result was the same as the longest ORF. An additional 157 full-length sequences (with disruptions) were generated from what had been previously considered fragments.

Removing Duplicates.

Even after conceptual translation, there were still some short fragments of OR sequences. These could be classified into two groups: fragments reaching the ends of short scaffolds or reaching the end of low-quality sequence regions (long stretches of ‘N’s) in large scaffolds (229 sequences), and short fragments not reaching any kind of ends in large scaffolds (61 sequences). Fragments in the first group might become full-length genes when more sequence information becomes available, whereas fragments in the second group were considered as pseudogenes with large deletions. For both groups, assuming the fragment was not mapped, if it could be matched to another longer mouse OR with >95% identity and the flanking DNA regions were almost identical, it was removed as a duplicate. Some full-length ORs were also removed because they were found on very short unmapped scaffolds (<<3 Kb) and they shared more than 98% identity with another full-length OR found on a longer mapped scaffold. It should be noted that all the ORs that were removed as duplicates need to be confirmed by future mapping data. Accordingly, all unmapped ORs that were removed as duplicates have been noted, and their final accurate mapping information will be used to determine if they truly are duplicates. In the rare cases where two fragments had an almost identical overlapping region both at the protein and DNA level, a long DNA sequence was assembled from the two short DNA sequences and conceptual translation was used to generate a long protein sequence. Eleven full-length sequences were generated this way. Finally, 1,296 non-duplicate sequences were obtained from a total of 1,405 sequences. Of these 1,296 sequences, 154 are fragments and 58 of those are pseudogenes with large deletions.

### Example 2

### Matches with known ORs

122 mouse ORs were collected from the ORDB, and 362 mouse ORs were collected from Genbank using ‘olfactory receptors’ or ‘odorant receptors’ as a keyword when searching mouse genes (all databases as of Jun. 25, 2001). The ORs from the public database were matched with our mouse OR database using FASTA3. For each OR from the public database the best hit was chosen, and the percentage of protein identity was used for further analysis. Similarly, human ORs and rat ORs were also matched with our mouse OR database.

### Example 3

### Matches with ORs from cDNA Sources and the Mouse EST Database

ORs labeled as originating from cDNA source material in the ORDB were selected, and each of these sequences was searched against our mouse OR database. Hits with >95% identity were considered as matches. The mouse EST database was downloaded from the NCBI server and BLASTN search was performed using every mouse OR DNA sequence as a query against the EST database. Hits with E-values <1e-100 were considered as matches.

### Example 4

### Identification of Mouse Olfactory Receptor Gene Family

1296 genes have been identified as set forth in SEQ ID NOS: 1-1296. This is by far the largest gene superfamily in the mammalian genome. About 80% (˜1000) of the ORs are potentially functional genes and 20% appear to be pseudogenes. Of the 1296 genes, 96 are partial sequences due to the current incompleteness of the Celera sequencing effort. This large number of ORs corresponds to previous predictions based on the number of glomeruli (i.e. sensory neuron targets) and from screening genomic phage libraries (Mombaerts, P. Molecular biology of odorant receptors in vertebrates. Annu Rev Neurosci 22, 487-509 (1999)). Proteins encoded by the identified MOR genes are as set forth by SEQ ID NOS: 1297-2592 in the attached sequence listing.

The Celera Mouse Genome claims greater than 99% coverage, but there are many low quality sequence regions (long stretches of ambiguous nucleotides), especially in long scaffolds. It remains possible that there are undiscovered OR sequences in these low quality sequence regions, as well as in remaining gaps. To determine how much of the mouse OR repertoire was covered, ORs accessible in public databases with the OR repertoire of the present invention. Over 90% of the mouse ORs in current public databases can be found in the OR repertoire of the present invention (Table 1). It should be noted, however, that some of the OR sequences in the public database could be PCR artifacts, i.e. “chimera receptors” due to recombination among highly related sequences, a phenomenon that has been documented (Glusman, G., Clifton, S., Roe, B. & Lancet, D. Sequence analysis in the olfactory receptor gene cluster on human chromosome 17: recombinatorial events affecting receptor diversity. Genomics 37, 147-60. (1996)). Newly identified nucleotide sequences are set forth by SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296 in the attached sequence listing. Proteins encoded by the above-identified new sequences are set forth as SEQ ID NOS: 1300-1302, 1302, 1308-1310, 1312-1323, 1326-1346, 1348, 1349, 1351-1365, 1368-1382, 1384-1388, 1393-1396, 1398-1417, 1419, 1421, 1423-1440, 1442-1448, 1450-1460, 1463, 1464, 1467-1469, 1471-1478, 1480-1487, 1489, 1490, 1492-1501, 1503, 1505-1510, 1512-1515, 1517-1521, 1523-1541, 1546-1548, 1550, 1552-1556, 1560-1566, 1569-1577, 1581-1588, 1590, 1592-1596, 1599, 1600, 1602-1609, 1611-1614, 1616, 1618-1625, 1627-1631, 1635-1648, 1650, 1651, 1653-1684, 1687, 1689-1722, 1724, 1725, 1728-1764, 1766-1782, 1784-1791, 1793-1803, 1805-1809, 1811-1821, 1823, 1824, 1828, 1829, 1831, 1834, 1836-1838, 1841-1852, 1854-1859, 1861-1872, 1874-1893, 1895-1914, 1916, 1920-1924, 1926, 1927, 1933-1952, 1955-1958, 1961, 1963-1971, 1973-1979, 1981-1993, 1997, 2000-2012, 2015-2034, 2036-2053, 2055-2062, 2064-2080, 2082, 2085-2092, 2096, 2097, 2099-2108, 2110-2130, 2132-2161, 2163-2222, 2224-2356, 2358-2390, 2392-2411, 2413-2422, 2425-2431, 2434-2440, 2442-2452, 2455-2461, 2464-2467, 2469-2471, 2473-2488, 2490-2492, 2494-2497, 2499, 2501-2523, 2525, 2528-2546, 2548, 2549, 2551-2554, 2556-2564, 2568-2570, 2572-2589, 2591 and 2592, respectively.

In addition, a few examples of non-OR sequences improperly labeled as ORs in the public database were found. In ORDB, the genes ORL248, ORL834, ORL844, ORL837 are not likely to be ORs (Skoufos, E. et al. Olfactory Receptor Database: a database of the largest eukaryotic gene family. Nucleic Acids Res 27, 343-5 (1999)). When compared to profile HMMs trained on human intact ORs they have large E-values (>30), while typical ORs have E-values <1-10. The OR repertoire of the present invention is believed to cover more than 90% of the whole mouse OR repertoire.

As a caveat, it should be noted that different mouse strains are known to have slightly different sequences for the same OR. The Celera mouse genome was assembled from four different strains (129X1/SvJ, 129S1/SvImJ, DBA/2J, and A/J). ORs in the assembled genome represent the consensus of the strains that had sequence available for that region. The real OR sequences in each strain might be slightly different, but generally should be >99% identical to the sequence in the current database. All of the sequences of the present invention were less than 98% identical with each other except in a very few cases where two very similar genes were unambiguously located at different genomic locations.

TABLE 1
Comparison of genome derived database with available mouse ORs
in public database.

| Database | ORs from Genbank | ORs from ORDB |
|---|---|---|
| Number of ORs | 362 | 122 |
| Matches (>95% identity) | 327 (90.3%) | 110 (90.2%) |
| Matches (>95% identity) | 280 (77.3%) | 93 (76.2%) |

### Example 5

### Phylogenetic Analysis of the OR Sequences

The 1296 mouse OR genes were aligned using ClustalX 1.81, which provides a graphical interface for the ClustalW multiple sequence alignment program. The resulting multiple alignment were used as input to phylogenetic analysis software program, PAUP*4.0 beta (Phylogenetic Analysis Using Parsimony) (Sinauer Associates, Inc., Sunderland, Mass.). The majority-rule consensus Neighbor Joining (NJ) tree from 1000 bootstraps was obtained from PAUP* requiring 48 hours running time on a 1 GHz Pentium III PC. Bootstrap methods of statistical analysis uses simulation to calculate standard errors, confidence intervals and significance tests. In this scenario, bootstrap is used to determine if a tree branch, or a cluster of genes, form a significant cluster. The tree comprises various clades, phylogenetic groups with shared characteristics and stemming from a common ancestor. OR families were determined from the largest clades that fulfilled two criteria: the clade had greater than 50% bootstrap support, and all members within that clade had at least 40% protein identity. In order to show a simplified tree, another tree with the consensus sequences of each family was built. Only intact full-length ORs from each family were used to generate the consensus sequence. The sequences from each family were aligned using ClustalW, after which a profile HMM was built upon the alignment, and the consensus sequence was generated from the profile HMM using the HMMER package. The ORs from families with only a single gene were used directly. All the consensus sequences were aligned and a NJ tree with 1000 bootstraps was built using ClustalX. The tree was rooted using human and mouse melanocyte stimulating hormone receptors (MSH-R), one of the GPCRs most closely related to ORs. The tree was plotted using Tree Explorer (http://evolgen.biol.metro-u.ac.jp/TE/TE_man.html).

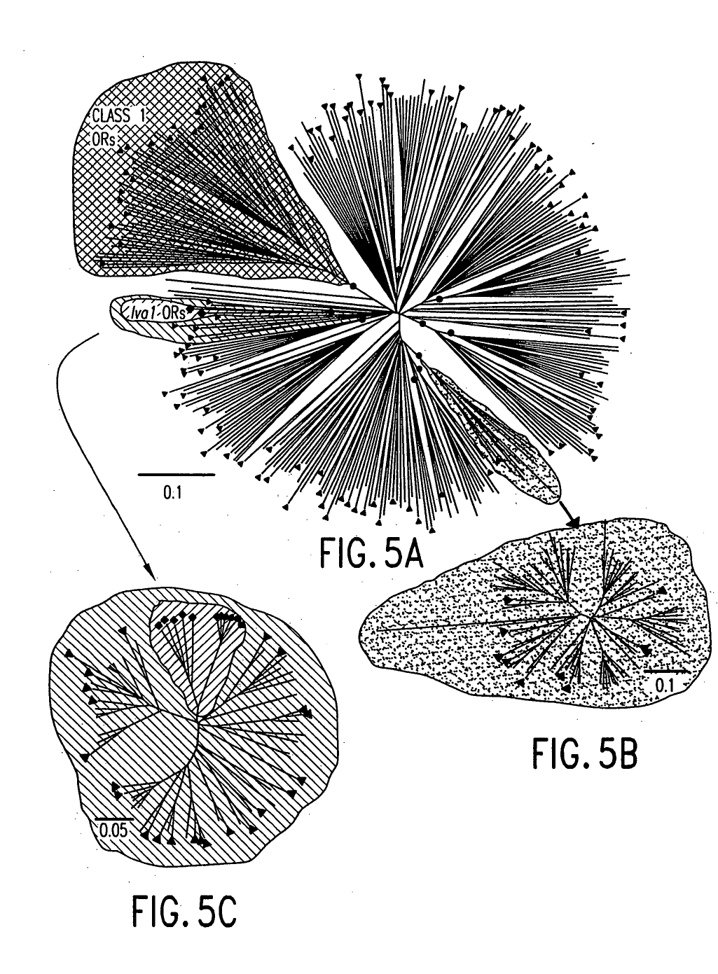

The same method was used to obtain human consensus sequences and to build a combined tree of all human and mouse OR families. The tree was unrooted and plotted using Tree Explorer. Selected ORs from human and mouse, examples of which are illustrated in FIG. 5b, FIG. 5c, were aligned and NJ trees with 1000 bootstraps were built using ClustalX.

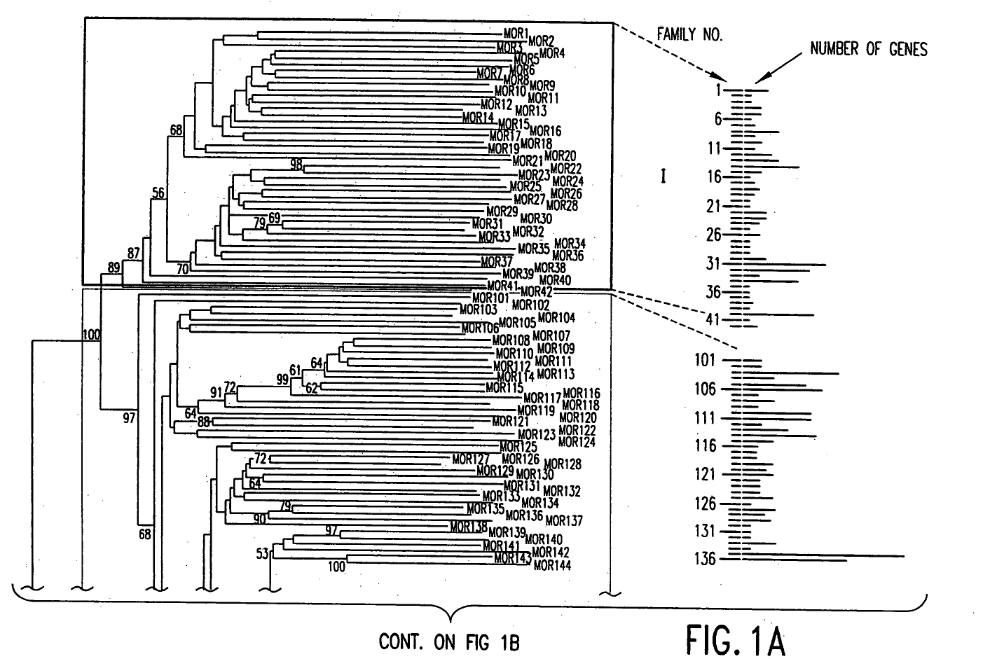

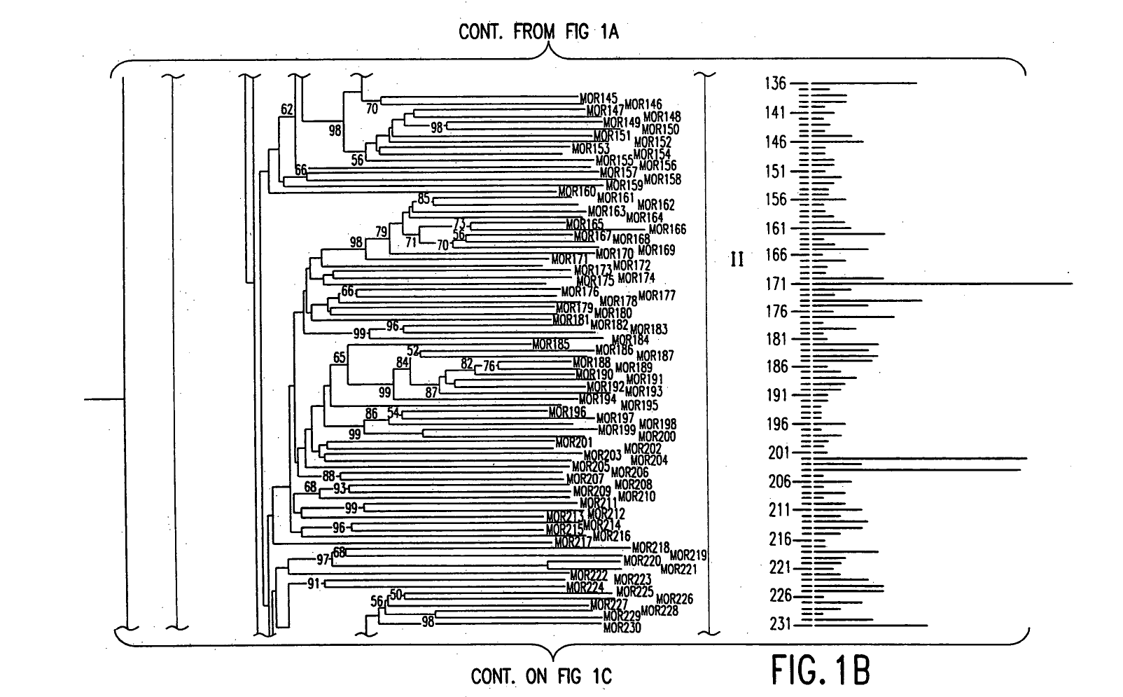

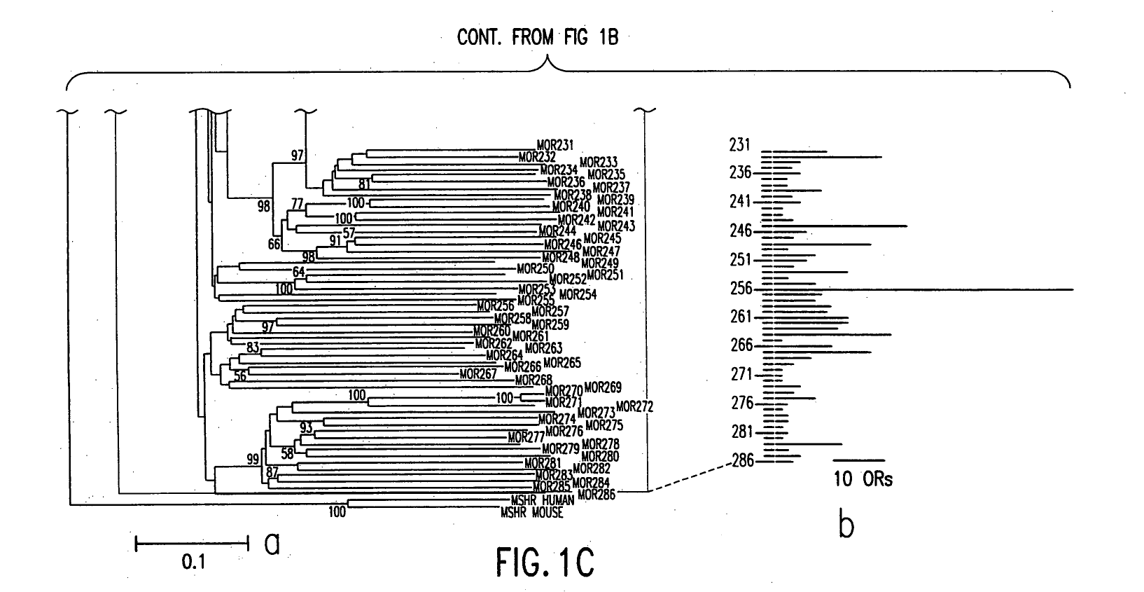

Based on the consensus tree, the ORs were classified into families using a rule whereby all family members must comprise a strong phylogenetic cluster, i.e., a reliable clade, generally possessing >50% bootstrap support, and should have more than 40% protein identity. By this definition mouse ORs could be classified into 228 families containing from 1 to 50 member genes. Since the complete tree of 1296 ORs cannot be clearly shown on one page, a phylogenetic tree using the consensus sequence for each family was built, as shown in FIG. 1a. FIG. 1a shows a phylogenetic tree of the consensus sequences of mouse OR families. The bootstrap values are shown at nodes with >50% support. The tree was rooted using human and mouse melanocyte stimulating hormone receptors (MSH-R). The OR sequences clearly separate into two broad classes, each with excellent bootstrap support. This is the same Class I and Class II distinction as reported previously for the human OR sequences (Glusman, G., Yanai, I., Rubin, I. & Lancet, D., “The complete human olfactory subgenome,” Genome Res 11, pp. 685-702 (2001)).

A classification for the mouse OR families was developed by the present invention, based on the phylogenetic tree, in which Class I OR families were given family numbers lower than 100 (currently 1-42), while Class II OR families were given family numbers higher than 100 (currently 101-286). If new families are discovered they can be given family numbers following the same rule. The number of genes in each family is shown in FIG. 1b. FIG. 1b depicts graphically Class I and Class II OR families, their respective family members, and the number of genes in each family.

The following nomenclature system for the mouse OR genes was used in the present invention: ‘MOR’ is used as a prefix, followed by the family name (Arabic number: 1-42, 101-286, as above), then a dash (-) followed by a number representing the individual gene within the family. The letter ‘P’ at the end of the name denotes that gene as possibly a pseudogene (see below for discussion on determining pseudogenes). For example, MOR1-1 is an intact family 1 OR belonging to Class I because the family number is less than 100; MOR185-9P is a pseudogene in family 185, which is a Class II family.

There are two existing nomenclature systems for human ORs. Lancet and colleagues proposed naming OR genes according to family (>40% amino acid identity) and subfamily (>60%) assignments (Glusman, G. et al., “The olfactory receptor gene superfamily: data mining, classification, and nomenclature,” Mamm Genome 11, pp. 1016-23 (2000)). Zozulya and colleagues introduced an alternative naming scheme mainly based on phylogenetic analysis, but also accounting for genomic location (Zozulya, S., Echeverri, F. & Nguyen, T., “The human olfactory receptor repertoire,” Genome Biol 2, pp. 0018.1-12 (2001)). The proposed nomenclature used herein is more similar to the second system, except genomic locations were not included.

### Example 6

### Pseudogenes

Of the 1296 OR genes, a significant number that had one or more disruptions in the coding region was identified, where a disruption could be an insertion, a deletion, a frame shift or a premature stop codon. However, all genes with disruptions are pseudogenes as some of the disruptions may be due to errors in the genomic sequences. There are examples of OR sequences that are known to be functionally expressed, but have disruptions in the Celera genome sequences. Besides possible sequencing errors, there are also polymorphisms in which one OR gene might have disruptions in some individuals, but are intact in others. (Glusman, G. et al. “Sequence, structure, and evolution of a complete human olfactory receptor gene cluster,” Genomics 63, pp. 227-45. (2000); Ehlers, A. et al. “MHC-linked olfactory receptor loci exhibit polymorphism and contribute to extended HLA/OR-haplotypes,” Genome Res 10, pp.1968-78. (2000); Younger, R. M. et al. “Characterization of clustered MHC-linked olfactory receptor genes in human and mouse,” Genome Res 11, pp. 519-30. (2001)).

In order to guide the estimation of pseudogenes, the available expression data for mouse ORs was obtained and compared to the OR database of the present invention, to the Olfactory Receptor Database (ORDB) (a world-wide web-accessible database that stores data on Olfactory Receptor-like molecules containing 96 mouse ORs from cDNA sources http://crepe.med.yale.edu/ORDB/HTML), and to the mouse EST database (NCBI). In total 174 ORs with at least one match to cDNA or EST sequences, suggesting functional expression. The percentage of expressed ORs was similar for genes with no disruptions and with one disruption (14% & 15% respectively), but this percentage dropped by almost half for ORs with two or more disruptions (8-10%), as indicated in Table 2a herein below. From this observation, it was estimated that many ORs with one disruption could be functional. On the other hand, ORs that have an intact reading frame could be pseudogenes if they lack important functional regions. Therefore ORs with zero or one disruption were checked for the presence of the conserved motifs found in all mammalian ORs (Mombaerts, P. “Molecular biology of odorant receptors in vertebrates,” Annu Rev Neurosci 22, pp.487-509 (1999)). Six ORs lacked one or more of these motifs and were therefore classified as pseudogenes. Accordingly in the nomenclature system used herein, full length ORs with two or more disruptions, partial length ORs with one or more disruptions, and ORs with less than one disruption, but missing conserved motifs, were labeled as pseudogenes as indicated in Table 2a herein below. Using this calculation, 260 of the 1296 ORs in mouse (20%) were classified as pseudogenes. If a more conservative approach was taken and only full-length genes with no disruptions were considered as functional genes, there would be only 873 functional genes, and 423 (33%) pseudogenes.

Since less than 10 pseudogenes have been previously reported in mouse (Xie, S. Y., Feinstein, P. & Mombaerts, P. “Characterization of a cluster comprising approximately 100 odorant receptor genes in mouse,” Mamm Genome 11, pp.1070-8 (2000); Lapidot, M. et al. “Mouse-Human Orthology Relationships in an Olfactory Receptor Gene Cluster,” Genomics 71, pp.296-306 (2001)), it was not expected that such a large portion of identified MOR genes would be pseudogenes. This 20% may be attributable to two factors. First, most OR sequences have been obtained from cDNA, which would miss untranscribed genes, and second, the common practice of using degenerate PCR for obtaining OR partial sequences would miss disruptions outside of the PCR-amplified region. In any case, the clearly large number of pseudogenes suggests that the OR superfamily undergoes rapid evolution, with new genes continuously being generated by duplication and mutation. For mouse ORs, it is expected that something less than the ˜1000 apparently intact genes may be functional in any individual animal. From limited data it appears that on average the cells expressing a given receptor target two glomeruli in the mouse olfactory bulb. Counting glomerular targets (˜1800) and dividing by 2 gives a rough estimate of 900 functional genes. (Mombaerts, P. “Molecular biology of odorant receptors in vertebrates,” Annu Rev Neurosci 22, pp.487-509 (1999)).

TABLE 2a
ORs with at least one match for expression data (from EST database or
a cDNA source).

No. of disruptions.

| | 0 | 1 | 2 | >2 |
|---|---|---|---|---|
| Total ORs | 904 | 177 | 70 | 145 |
| ORs with match | 128 | 27 | 7 | 12 |
| (ORs w/match)/total | 14% | 15% | 10% | 8.3% |

TABLE 2b
Number of intact genes and pseudogenes from each group of ORs
according to the length and number of disruptions.

| | | | | Full | | | Partial |
|---|---|---|---|---|---|---|---|
| | | Full | Full | length, | Partial | Partial | length, |
| | | length, | length, | 2 or | length, | length, | 2 or |
| | | no | 1 | more | no | 1 | more |
| | Total | dsrpt. | disrpt. | disrpt. | dsrpt. | disrpt. | disrpt. |
| All OR | 1296 | 875 | 138 | 129 | 29 | 39 | 86 |
| genes | | | | | | | |
| Intact | 1036 | 873 | 134 | 0 | 29 | 0 | 0 |
| genes | | | | | | | |
| Pseudo- | 260 | 2* | 4* | 129 | 0 | 39 | 86 |
| genes | | | | | | | |

*These ORs were labeled as pseudogenes because they lacked one or more of the OR signature sequences.

### Example 7

### Genomic Distribution of MOR genes

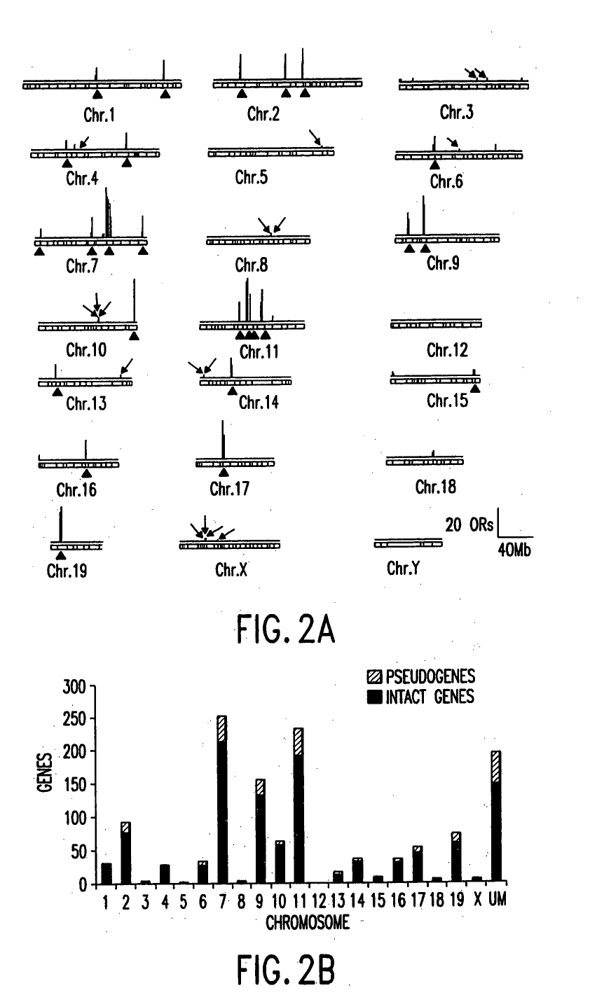

OR genes were distributed mainly in clusters on all mouse chromosomes except 12 and Y. Out of the 1296 ORs, 1103 were mapped to 18 chromosomes (the rest were on currently unmapped sequence regions), as shown in FIGS. 2a and 2b. FIGS. 2a and 2b show the chromosomal distribution of Mouse OR Genes. In FIG. 2a, mouse chromosomes are drawn according to the Celera scaffold assembly. Scaffolds with unknown directions are in gray. The cytogenetic map of each chromosome is shown under the scaffold assembly in scale (from Animal Genome Database, http://ws4.niai.affrc.go.jp/dbsearch2/mmap/mmap.html). The number of ORs per 500 Kb are depicted as bars on each chromosome. OR clusters are indicated by arrow heads. Arrows indicate isolated single genes on the chromosomes that are not easily visualized. Scaffolds on chromosome Y are not yet mapped in the Celera mouse genome, but based on results from human and other species, it is unlikely that there will be ORs on chromosome Y.

Chromosome 7 housed the largest number of ORs (252), while chromosome 11 (190 ORs) and chromosome 9 (131 ORs) were the second and the third most populous. In contrast, chromosomes 3 and 8 had only 2 ORs apiece, and chromosome X had only 4 ORs (FIG. 2b). The graph in FIG. 2b shows the number of intact genes (dark) and pseudogenes (shaded) on each chromosome. UM (‘UnMapped’) represents ORs from currently unmapped scaffolds.

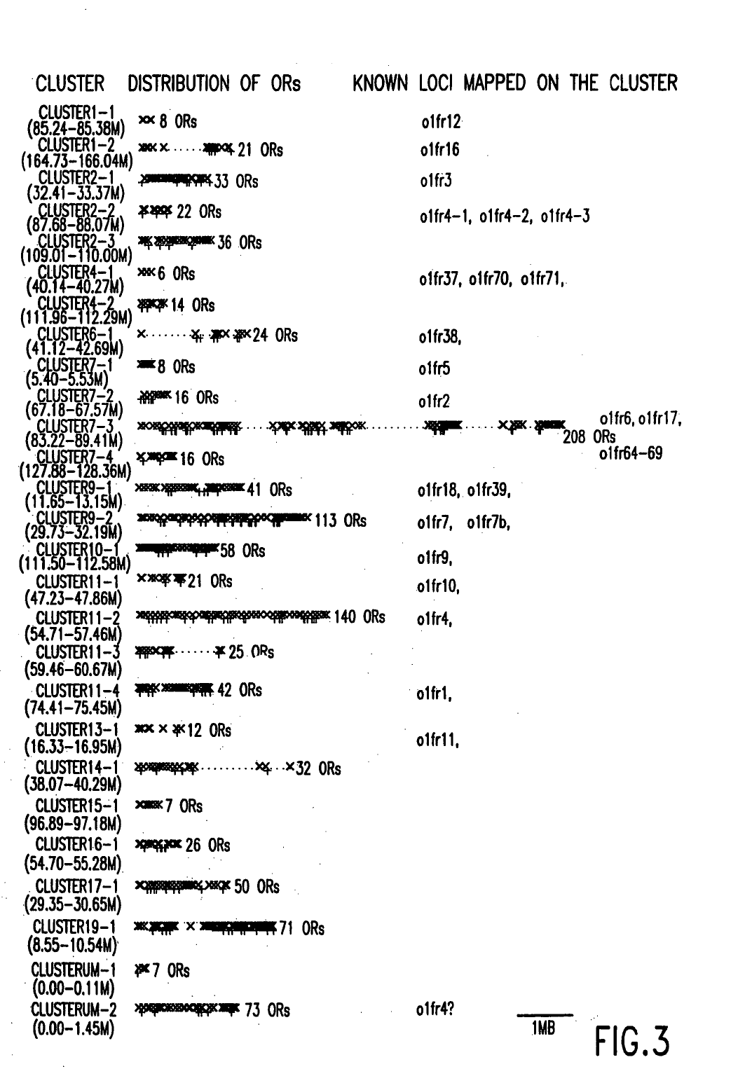

Gene clusters were determined using the same definition as in the human OR genome: clusters contain more than 5 genes, none of which are separated by more than 1 Mb. Twenty-seven mouse OR clusters were identified, including two on currently unmapped scaffolds which could turn out to be part of existing clusters, which contain a total of 1130 OR genes, as shown on FIG. 3. The frequency of ORs in these clusters was high, ranging from 18 to 66 Kb per OR (average=29 Kb per OR). OR clusters were named by their chromosome number (1-19, and UM for “UnMapped”), and the index number of the cluster on its chromosome. Interestingly, ORs from the same family tended to locate near each other, forming ‘subclusters’. There were some ORs not located in any of the clusters; however, they still tended to locate as “miniclusters” (i.e., they consist of fewer than 5 genes together), e.g., ORs on chromosome 18 shown in FIG. 2a. Isolated single ORs were rarely seen; however, one was located in the middle of chromosome X.

In spite of the density of OR genes, non-OR genes were regularly found within the OR clusters. Only 5 small OR clusters (1-1,4-1, 4-2, 7-1, UM-1) were completely free of non-OR genes; all other clusters had some non-OR genes distributed within them. Interestingly, viral coat proteins (Gag, ENV, and Pol polyproteins) were often found in OR clusters. Notably, the retrovirus-related Gag or Gag-related proteins could be found in 15 out of the 27 OR clusters, presented in one to eight copies. The density of Gag or Gag-related proteins was twice as high in OR clusters than in the rest of the genome. The presence of viral coat proteins in OR clusters suggests a possible viral-based mechanism of gene duplication and relocation. ORs, like many mammalian GPCRs, are generally intronless, and one theory attributes this to a retroviral mediated duplication of the family (Brosius, J. “Many G-protein-coupled receptors are encoded by retrogenes,” Trends Genet 15, pp.304-5. (1999); Gentles, A. J. & Karlin, S. “Why are human G-protein-coupled receptors predominantly intronless?” Trends Genet 15, pp. 47-9. (1999)).

The present invention is useful to address whether if phylogenetically related ORs are also located close to one another. To determine this, OR pairs that were at least 60% identical at the protein level were identified and their relative chromosomal locations were determined. It was found that 1176 ORs had another OR at least 60% identical to it, but because 239 of these pairs had at least one OR not mapped to a chromosome location, only the remaining 937 were analyzed. In 918 (98.0%) of these 937 cases, the two ORs were located on the same chromosome within a median distance of 44 Kb. In 55.3% (518/937) cases, the two ORs were either adjacent to each other, or were separated by only one other OR. The close proximity of highly related ORs suggests significant local duplication as another mechanism of OR family expansion, in addition to large scale duplication of OR clusters.

A few mouse OR loci have been genetically mapped to chromosomal locations (Copeland, N. G. et al. “A genetic linkage map of the mouse: current applications and future prospects,” Science 262, pp. 57-66. (1993); Sullivan, S. L., Adamson, M. C., Ressler, K. J., Kozak, C. A. & Buck, L. B. “The chromosomal distribution of mouse odorant receptor genes,” Proc Natl Acad Sci USA 93, pp. 884-8 (1996); Carver, E. A., Issel-Tarver, L., Rine, J., Olsen, A. S. & Stubbs, L. “Location of mouse and human genes corresponding to conserved canine olfactory receptor gene subfamilies,” Mamm Genome 9, pp. 349-54. (1998); Strotmann, J. et al. “Small subfamily of olfactory receptor genes: structural features, expression pattern and genomic organization,” Gene 236, pp. 281-91. (1999); Asai, H. et al. “Genomic structure and transcription of a murine odorant receptor gene: differential initiation of transcription in the olfactory and testicular cells,” Biochem Biophys Res Commun 221, pp. 240-7. (1996); Bulger, M. et al. “Conservation of sequence and structure flanking the mouse and human beta-globin loci: the beta-globin genes are embedded within an array of odorant receptor genes,” Proc Natl Acad Sci USA 96, pp. 5129-34. (1999); Zheng, C., Feinstein, P., Bozza, T., Rodriguez, I. & Mombaerts, P. “Peripheral olfactory projections are differentially affected in mice deficient in a cyclic nucleotide-gated channel subunit,” Neuron 26, pp. 81-91. (2000)). These loci were matched to the OR database of the present invention using either their OR sequences or molecular marker sequences. The same chromosome locations were found for all of the known mouse OR loci except for olfr4. FIG. 3 shows a schematic diagram demonstrating the distribution of OR Clusters. Detailed distribution of ORs in each of the 27 clusters are shown. Intact genes are labeled as ‘x’, and pseudogenes are labeled as ‘+’. It can be seen that similar ORs tend to locate together, forming patches in the figure. All the Class I ORs are located in cluster 7-3. Most families are located in a restricted area in one cluster.

Although only one or a few ORs were previously known for each locus, they are now mapped to OR clusters with dozens of ORs. Not surprisingly, it quite often happened that multiple known loci actually mapped to different locations within the same cluster.

Two OR clusters have recently been studied in detail. Eighteen mouse OR genes were found in olfr17, which matches to one subcluster (87.5M to 87.9M) of 25 ORs (SEQ ID NOS: 65, 93-96, 356, 536-544, 758, 828, 866, 867, 937, 1127, 1128, 1238) in cluster 7-3 (Lane, R. P. et al. “Genomic analysis of orthologous mouse and human olfactory receptor loci,” Proc Natl Acad Sci USA 98, pp. 7390-5. (2001)). Forty-two ORs have been identified in olfr7, and the total number of ORs in this cluster was estimated to be around 100 (Xie, S. Y., Feinstein, P. & Mombaerts, P. “Characterization of a cluster comprising approximately 100 odorant receptor genes in mouse,” Mamm Genome 11, pp. 1070-8 (2000)). This group matches to cluster 9-2 in the ORDB of the present invention. In fact, this cluster has 113 ORs.

Olfr4 was previously mapped to chromosome 2 and has three known genes. However, one gene mapped to cluster 11-2 and two genes to an unmapped scaffold, UM-2 that was also likely to be on chromosome 11. This discrepancy of the location of olfr4 may require additional data to be resolved.

### Example 8

### Candidate OR Cluster for a Specific Anosmia to Isovaleric Acid

Several naturally occurring specific anosmias (the inability to detect an odor) have been reported in human and mouse (Griff, I. C. & Reed, R. R. “The genetic basis for specific anosmia to isovaleric acid in the mouse,” Cell 83, pp. 407-14. (1995)). C57BL/6J and C57BL/10J mice have a specific anosmia (or more precisely, a narrowly limited hyposmia) to isovaleric acid, and this defect appears to have a peripheral source (Wysocki, C. J., Whitney, G. & Tucker, D. “Specific anosmia in the laboratory mouse,” Behav Genet 7, pp. 171-88. (1977); Wang, H. W., Wysocki, C. J. & Gold, G. H. “Induction of olfactory receptor sensitivity in mice,” Science 260, pp. 998-1000. (1993)). This anosmia is recessive and two loci responsible for the defect have been mapped, Iva1 on chromosome 4, and Iva2 on chromosome 6 (Griff, I. C. & Reed, R. R. “The genetic basis for specific anosmia to isovaleric acid in the mouse,” Cell 83, pp. 407-14. (1995)). Based on the adjacent molecular markers, the present invention has located Iva1 at 110.46M-112.23M on the reference axis of chromosome 4, a location that matches very well with OR cluster 4-2 (111.96M-112.29M on chromosome 4). The 14 ORs (SEQ ID NOS: 279, 280, 425, 426, 740, 792, 850, 853, 887, 888, 1159, 1193, 1203, and 1212) in this cluster were from families 258 and 259, two closely related families that form a clade with 97% bootstrap support value. Two other unmapped ORs (SEQ ID NOS: 1164 and 1107) also belong to these two families. Because highly related sequences were usually located near each other, it was likely that the two unmapped ORs probably were also part of cluster 4-2. No other ORs from any other position fell into these two families. Thus, the 16 genes in families 258 and 259 may be considered Iva1 ORs. The other locus, Iva2, was mapped to 136.1M to 140.9M of chromosome 6, but no OR sequences were found in this region. There is a sequence gap in the Celera mouse genome in this region, so it remains possible there are ORs in this gap. However, considering its weak correlation with the anosmia, and the fact that one of the markers (D6MIT201) used to located Iva2 actually maps to the Iva1 locus in the Celera mouse genome, Iva2 is not likely to be the true locus for isovaleric acid anosmia. The most likely cause of the anosmia in C57BL/6J and C57BL/10J mice would appear to be the loss of OR proteins in cluster 4-2. Since the strains used in Celera mouse genome database are osmic animals, the Iva1 locus of the anosmic strains may be useful to determine the cause underlying the loss of these OR proteins.

This result also suggests that some or all the Iva1 ORs bind isovaleric acid with high affinity. Anosmic strains of mice respond to isovaleric acid at a higher threshold than mice in osmic strains, suggesting that the defect involves a loss of high affinity receptors (Wang, H. W., Wysocki, C. J. & Gold, G. H. Induction of olfactory receptor sensitivity in mice. Science 260, 998-1000. (1993)). Of the 16 Iva1 OR genes, 6 intact genes were in family 258 sharing 50-80% protein identity, and 7 intact genes plus 3 pseudogenes were in family 259, sharing 80-95% protein identity. Genes from the two families shared 35-50% protein identity, suggesting they may have different ligand binding profiles. Functional studies of these two families may reveal which family, or which ORs, are responsible for recognizing isovaleric acid at high affinity.

Interestingly, when the Iva1 ORs were compared with human ORs, no intact human OR fell into the same clade as the Iva1 ORs (FIG. 5c). At least from the available human data, no orthologs of Iva1 ORs are present in humans, which suggests that humans lack the high affinity receptors for isovaleric acid. In animal behavior assays in which isovaleric acid was diluted in buffered solution adjusted to its pH, anosmic animals could detect isovaleric acid only at concentrations higher than 10⁻⁵ M, while osmic strains were sensitive to isovaleric acid at concentrations as low as 10⁻⁷ M (Griff, I. C. & Reed, R. R., “The genetic basis for specific anosmia to isovaleric acid in the mouse,” Cell 83, pp. 407-14. (1995)). According to Flavor-Base Pro (Leffingwell & Associates, Canton, Ga.), the human flavor threshold for isovaleric acid in water is 120-700 ppb, which is equivalent to 1.2-6×10⁻⁶ M, a number between the thresholds of anosmic and osmic mice. Strictly controlled experiments under the same conditions as those used in animal assays would be required to determine the precise human threshold for isovaleric acid.

### Example 9

### Class I ORs

Class I ORs were first identified in fish and they separate clearly in the phylogenetic tree from the classical, mammalian specific Class II ORs (Ngai, J., Dowling, M. M., Buck, L., Axel, R. & Chess, A., “The family of genes encoding odorant receptors in the channel catfish,” Cell 72, pp. 657-66. (1993)). There were 147 of the ‘Fish-like’ Class I ORs in the mouse OR subgenome, and 120 of them were potentially functional, as indicated in FIGS. 1a and 1b. All of the Class I ORs were located in a single huge cluster on chromosome 7 (cluster 7-3, shown in FIG. 3). This confirms that Class I ORs are prevalent in the mammalian genome, and suggests that they may play important roles in mammalian olfaction.

Interestingly, 11 of the 14 ORs identified in a recent study as having aliphatic odor ligands belong to Class I. (Malnic, B., Hirono, J., Sato, T. & Buck, L. B., “Combinatorial receptor codes for odors,” Cell 96, pp. 713-23. (1999)). In that study, odorant compounds were applied to dissociated olfactory neurons plated on coverslips, and responses were monitored by calcium imaging. The large portion of Class I ORs found in this study is unusual (11/14 compared to their 12% occurrence in the whole OR repertoire), suggesting that the experimental design or the compounds used in the study may favor activating olfactory neurons expressing Class I ORs. The former can be eliminated since similar experiments using different odorant compounds did not isolate a large proportion of Class I ORs (Touhara, K. et al., “Functional identification and reconstitution of an odorant receptor in single olfactory neurons,” Proc Natl Acad Sci USA 96, pp. 4040-5. (1999); Kajiya, K. et al., “Molecular Bases of Odor Discrimination: Reconstitution of Olfactory Receptors that Recognize Overlapping Sets of Odorants,” J Neurosci 21, pp. 6018-25. (2001)). Therefore it seems likely that the aliphatic compounds used in this study were mostly Class I OR specific ligands. It is worth noting that these compounds were acids and alcohols, which are relatively hydrophilic compared to many other odorant compounds.

Class I ORs are related to Fish ORs, which are expected to bind water-soluble compounds. In frog Class I ORs are activated by water-soluble odorants, whereas Class II ORs are activated by volatile compounds. (Mezler, M., Fleischer, J. & Breer, H., “Characteristic features and ligand specificity of the two olfactory receptor classes from Xenopus laevis,” J Exp Biol 204, pp. 2987-97. (2001)). In mammals, however, water-soluble compounds generally are not strong odorants, and mammalian Class I ORs diverge significantly from the fish ORs or frog Class I ORs. Class I ORs in mammals may have evolved to recognize volatile compounds, although they are still more sensitive to relatively hydrophilic compounds, while Class II ORs may favor more hydrophobic compounds.

### Example 10

### Global View of Sequences of ORs

Clustal X 1.81 was used for multiple alignments of full length intact Class I ORs and Class II ORs. The alignments were manually edited. Gap positions present in more than 98% of the sequences were deleted. These gap positions are deleted because they are in only present in a very small portion of the genes and they are not characteristic of the whole family. Sequence logos were generated using a known web-based program developed by Jan Gorodkin (www.cbs.dtu.dk/gorodkin/appl/plogo/html). Sequence logos are a method of displaying consensus sequences that reflect the relative frequency of the bases and information content at various positions along a sequence. (J. Gorodkin, L. J. Heyer, S. Brunak and G. D. Stormo., Comput. Appl. Biosci., Vol. 13, No. 6 pp. 583-586, 1997, and T. D. Schneider and R. M. Stephens., “Sequence logos: a new way to display consensus sequences,” Nucleic Acids Research, Vol. 18, No. 20, pp. 6097-6100, 1990). The sequence, or structure, logo comprises a sequence part and a structure/basepair part. The height of the sequence information part reflects the relative entropy between the observed fractions of a given symbol and the respective a priori probabilities, where the constraint that the a priori “probability” of the gap always is one. The a priori probabilities for amino acids sum to one. If sufficiently multiple gaps are present at a given position, negative “information” may occur. The height of each symbol can be displayed to demonstrate that it is proportional to its frequency, or that the height is in proportion to the fraction of the observed frequency and the expected (a priori) frequency. When an amino acid appears less than expected, it appears displayed upside-down.

The secondary structure prediction was based on the PredictProtein Server results on consensus sequences generated by the HMMER package.

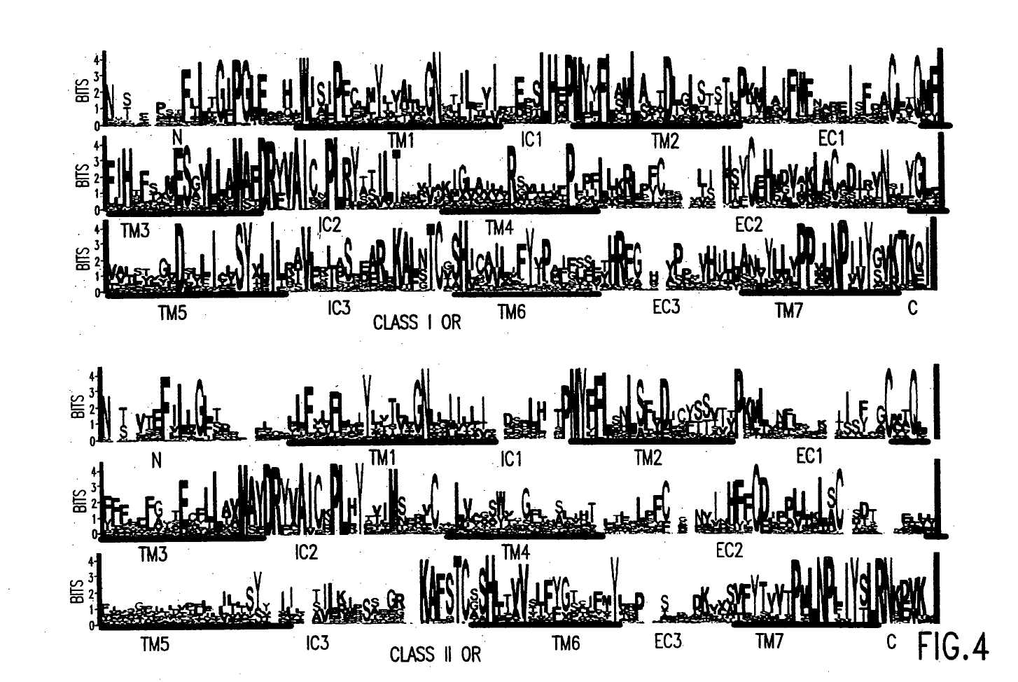

‘Logo’ views of OR sequences of the two Classes have been generated in order to facilitate visual sequence comparisons, as shown in FIG. 4. Predicted transmembrane (TM), intracellular (IC), and extracellular (EC) regions are depicted on FIG. 4. The very terminal sequences are removed to avoid length heterogeneity and no significant sequence conservation was found in these regions. The height of each amino acid is proportional to its frequency of occurrence. The large number of genes tends to reduce the conservation of any given residue. Nonetheless, the characteristic OR sequences can be easily seen. FIG. 4 shows that both Classes have the characteristic olfactory receptor specific regions, e.g. MAYDRYVAIC in TM3-IC2 (SEQ ID NO. 2593), FSTCSSH in IC3-TM6 (SEQ ID NO. 2594), and the three conserved cysteines in EC2. However, there were also regions that were quite distinct between the two Classes. For example, the ‘MAYDRY’ (SEQ ID NO. 2595) motif was more often ‘MAFDRY’ (SEQ ID NO. 2596) in Class I ORs; there were 3 conserved prolines in TM7 of Class I ORs, but only 2 prolines in Class II ORs; a highly conserved region starting from the middle of IC2 to the middle of TM4 (M . . . C . . . Lv . . . sW) was present in virtually all Class II ORs but absent in Class I ORs.

Transmembrane domains 4 and 5, and to lesser extent TM3, have been shown to be the most variable regions. (Pilpel, Y. & Lancet, D., “The variable and conserved interfaces of modeled olfactory receptor proteins,” Protein Sci 8, pp. 969-77. (1999)). This is also clear in the logo view, where fewer conserved residues appeared in these regions. Through the analyses of conserved and variable regions of the mammalian ORs, the present invention may provide key regions that may be instructive for functional studies of ORs in particular, and GPCRs generally.

### Example 11

### Comparison with other Mammalian Species

The most closely related species to mouse with numerous sequences available in the ORDB is the rat. Isolated examples have suggested that the two species might have very similar OR repertoires (e.g. rat I7 and mouse I7 receptors are 95% identical.) (Krautwurst, D., Yau, K. W. & Reed, R. R. Identification of ligands for olfactory receptors by functional expression of a receptor library. Cell 95, 917-26 (1998)). The present invention shows that the two species do indeed have similar OR repertoires, with 90% of the 65 rat ORs having a mouse ortholog with more than 80% identity. However, 45% of the rat ORs did not have a mouse ortholog with >90% identity, indicating that there are significant differences between the two repertoires as well (Table 3). Identifying precisely where the sequences diverge may provide interesting hints regarding functional differences between receptors in the two species.

TABLE 3
Cross-species matches with mouse OR database
Intact human ORs (347) and rat ORs (65) were compared with
mouse OR database, the protein identity of the closest match
of each gene were used for calculation.

| | >40% | >60% | >80% | >90% | >95% |
|---|---|---|---|---|---|
| Species | identity | identity | identity | identity | identity |
| human | 100% | 93% | 58% | 5% | 0.3% |
| | (347/347) | (323/347) | (200/47) | (18/347) | (1/347) |
| rat | 100% | 98% | 91% | 55% | 17% |
| | (65/65) | (64/65) | (59/65) | (36/65) | (11/65) |

More distant from mouse, humans show significant differences in the OR repertoire. Although all intact human OR genes had mouse orthologs of more than 40% identity, only 59% had an ortholog with >80% identity, and the number drops to 4% for ORs with a mouse ortholog of >90% identity (Table 3). One OR gene (OR93) has been identified in a few species of apes; it has an ortholog with 96% identity in human (OR5G3P), but the closest match in mouse had only 80% identity. A few dog ORs seem to fall somewhere in between human and mouse, showing around 80% identity to either species.

Since the complete human OR repertoire is available by the present invention, a comprehensive phylogenetic analysis of the human and mouse OR subgenomes is possible. Some 347 human intact ORs have been classified into 119 families 9, and 119 consensus sequences were produced for each of the human OR families. This permitted us to build a phylogenetic tree of consensus sequences of all human and mouse OR families as shown in FIG. 5a. FIG. 5a shows an unrooted phylogenetic tree of the consensus sequences of mouse and human OR families. Consensus sequences from each human and mouse OR family were used to build a Neighbor Joining tree with 1000 bootstraps. Human OR families are indicated by filled triangles A few groups, or clades, with high (>90%) bootstrap value are labeled by dots. The Class I OR families are shaded in gray and they form a group with more than 90% bootstrap value. In this two-species tree, it can be seen that although the mouse genome possessed nearly triple the number of intact ORs, the overall structures of the two OR repertoires were similar, and they covered more or less the same ‘receptor space’. While the mouse generally had more ORs in each broad branch, humans did not appear to have lost any of the major branches.

Also in FIG. 5a, a group with high bootstrap value and including mostly mouse OR families is shaded and plotted in detail in FIG. 5b. This group contains 11 mouse OR families and only 1 human family. Although there are only 10 intact human ORs in this group, they do not form one tight subgroup, but rather intermingle with the 76 intact full-length mouse ORs covering more than half of the subgroups. Even in some groups predominated by mouse families, human ORs in those groups still intermingled with the mouse ORs and covered a relatively broad space. This suggests that the human olfactory system has probably retained the ability to recognize a broad, if perhaps less discriminating, spectrum of chemicals while using one-third the number of ORs as in mouse.

Also in FIG. 5a, the Iva1 ORs, i.e. Mouse OR families 258 and 259, are shaded, indicated by open diamonds, and plotted in detail in FIG. 5c. These groups were present only in mouse and show a high bootstrap value (FIG. 5c). These exclusive mouse groups were not common and were typically small. Given that olfactory coding is likely to be primarily combinatorial, using numerous ORs to recognize an odor compound, the missing ORs in humans are more likely to alter sensitivity or discrimination, but not the range of detectable compounds.

1. A nucleic acid molecule comprising a nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

2. The nucleic acid molecule of claim 1, wherein said nucleic acid molecule is selected from the group consisting of RNA and DNA.

3. A vector comprising a nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

4. The vector of claim 3 wherein the vector is selected from the group consisting of a virus and a plasmid.

5. The vector of claim 4 wherein the nucleotide sequence is operably linked to a promoter sequence.

6. A host cell transformed with an expression vector comprising a nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

7. A polypeptide encoded by a nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

8. A polypeptide comprising an amino acid sequence selected from the group consisting of SEQ ID NOS: 1300-1302, 1302, 1308-1310, 1312-1323, 1326-1346, 1348, 1349, 1351-1365, 1368-1382, 1384-1388, 1393-1396, 1398-1417, 1419, 1421, 1423-1440, 1442-1448, 1450-1460, 1463, 1464, 1467-1469, 1471-1478, 1480-1487, 1489, 1490, 1492-1501, 1503, 1505-1510, 1512-1515, 1517-1521, 1523-1541, 1546-1548, 1550, 1552-1556, 1560-1566, 1569-1577, 1581-1588, 1590, 1592-1596, 1599, 1600, 1602-1609, 1611-1614, 1616, 1618-1625, 1627-1631, 1635-1648, 1650, 1651, 1653-1684, 1687, 1689-1722, 1724, 1725, 1728-1764, 1766-1782, 1784-1791, 1793-1803, 1805-1809, 1811-1821, 1823, 1824, 1828, 1829, 1831, 1834, 1836-1838, 1841-1852, 1854-1859, 1861-1872, 1874-1893, 1895-1914, 1916, 1920-1924, 1926, 1927, 1933-1952, 1955-1958, 1961, 1963-1971, 1973-1979, 1981-1993, 1997, 2000-2012, 2015-2034, 2036-2053, 2055-2062, 2064-2080, 2082, 2085-2092, 2096, 2097, 2099-2108, 2110-2130, 2132-2161, 2163-2222, 2224-2356, 2358-2390, 2392-2411, 2413-2422, 2425-2431, 2434-2440, 2442-2452, 2455-2461, 2464-2467, 2469-2471, 2473-2488, 2490-2492, 2494-2497, 2499, 2501-2523, 2525, 2528-2546, 2548, 2549, 2551-2554, 2556-2564, 2568-2570, 2572-2589, 2591 and 2592.

9. An isolated antibody that is capable of binding to a polypeptide encoded by nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

10. A method for identifying compounds useful for treating anosmia comprising (a) identifying a candidate compound by its ability to bind to a polypeptide encoded by nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296; (b) introducing the candidate compound to an anosmic mouse; and (c) determining whether the mouse detects isovaleric acid at a concentration less than 10⁻⁵ M after the candidate compound has been introduced, wherein the ability of the mouse to detect isovaleric acid at a concentration less than 10⁻⁵ M indicates that the compound is useful for the treatment of anosmia.

11. A method for identifying a compound for pest control comprising (a) identifying a candidate compound by its ability to bind to a polypeptide encoded by nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391,.393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926,928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296; (b) introducing the candidate compound in the vicinity of an animal; and (c) examining the behavior of the animal to determine whether the animal moves towards the candidate compound wherein the movement towards the candidate compound indicates that the candidate compound attract pests.

12. A method for identifying a compound for pest control comprising (a) identifying a candidate compound by its ability to bind to a polypeptide encoded by nucleotide sequence selected from the group consisting of SEQ ID NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296; (b) introducing the candidate compound in the vicinity of an animal; and (c) examining the behavior of the animal to determine whether the animal moves away from the candidate compound wherein the movement away from the candidate compound indicates that the candidate compound repels pests.

13. A method for identifying a compound which inhibits a mouse OR comprising (a) contacting a candidate compound with a mouse OR protein under condition that allow the mouse OR protein to interact with the target protein and (b) monitoring for contact between the mouse OR protein and the candidate compound using an assay wherein said mouse protein is encoded by a nucleotide sequence selected from the group consisting of SEQ NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

14. A method for identifying a compound that stimulates a mouse OR comprising (a) contacting a candidate compound with a mouse OR protein under condition that allow the mouse OR protein to interact with the target protein and (b) monitoring for contact between the mouse OR protein and the candidate compound using an assay wherein said mouse protein is encoded by a nucleotide sequence selected from the group consisting of SEQ NOS: 4-6, 8, 12-14, 16-27, 30-50, 52, 53, 55-69, 72-86, 88-92, 97-100, 102-121, 123, 125, 127-144, 146-152, 154-164, 167, 168, 171-173, 175-182, 184-191, 193, 194, 196-205, 207, 209-214, 216-219, 221-225, 227-245, 250-252, 254, 256-260, 264-270, 273-281, 285-292, 294, 296-300, 303, 304, 306-313, 315-318, 320, 322-329, 331-335, 339-352, 354, 355, 357-388, 391, 393-426, 428, 429, 432-468, 470-486, 488-495, 497-507, 509-513, 515-525, 527, 528, 532, 533, 535, 538, 540-542, 545-556, 558-563, 565-576, 578-597, 599-618, 620, 624-628, 630, 631, 637-656, 659-662, 665, 667-675, 677-683, 685-697, 701, 704-716, 719-738, 740-757, 759-766, 768-784, 786, 789-796, 800, 801, 803-812, 814-834, 836-865, 867-926, 928-1060, 1062-1094, 1096-1115, 1117-1126, 1129-1135, 1138-1144, 1146-1156, 1159-1165, 1168-1171, 1173-1175, 1177-1192, 1194-1196, 1198-1201, 1203, 1205-1227, 1229, 1232-1250, 1252, 1253, 1255-1258, 1260-1268, 1270-1272, 1274-1293, 1295 and 1296.

15. A data mining method for identifying gene family members comprising
(a) providing reference organism gene family sequences, candidate organism gene family sequences and query sequences;
(b) performing a low stringency TBLASTN search using the query sequence to obtain a pool of potential candidate sequences from a candidate organism database;
(c) building a profile Hidden Markov Model (HMM) using sequences from the reference and candidate organism to generate a profile HMM;
(d) comparing the pool of potential candidate sequences with the profile HMM to generate a pool of candidate sequences; and
(e) determining whether the candidate sequences obtained in step (d) are gene family members.

16. A method for identifying OR sequences comprising
(a) providing reference organism gene family sequences, candidate organism gene family sequences and query sequences;
(b) performing a low stringency TBLASTN search using the query sequences obtain a pool of potential candidate OR sequences;
(c) building a profile Hidden Markov Model (HMM) using the OR sequences from a reference organism and a candidate organism to generate a profile HMM;
(d) comparing the pool of potential candidate OR sequences with the profile HMM to generate a pool of candidate OR sequences; and
(e) determining whether the candidate sequences obtained in step (d) are OR gene family members.
